(19) United States
(12) Patent Application Publication
(10) Pub. No.: US 2006/0030758 A1
(43) Pub. Date: Feb. 9, 2006

(54) Mypersonallab

(75) Inventor: Melanie S. Amaral, Ceres, CA (US)

Correspondence Address: Melanie S. Amaral, P.O. Box 757, Ceres, CA, 95307, US

(73) Assignee: Francotyp-Postalia AG & Co. KG

(21) Appl. No.: 11/032,669

(22) Filed: Sep. 15, 2005

Related U.S. Application Data
(60) Provisional application No. 60/599,969, filed on Aug. 9, 2004.

(51) Int. Cl. A61B 5/00

(52) U.S. Cl. 600/300

(57) ABSTRACT

Many physicians, including endocrinologist and internist, have expressed great interest in such a monitoring devise. In a medical office, a doctor could use MyPersonalLab to determine the quantitative value of various blood electrolytes to diagnose and treat a multitude of metabolic diseases much faster, and therefore alleviating a patient's suffering that much faster. At home, the average individual could use MyPersonalLab to monitor his/her own disease state or medical condition. If they detected a value that their doctor previously determined was abnormal, they could then alert their physician of the concern. One might think that the clinical laboratory establishment would be highly opposed to such a devise, citing that it would reduce their traffic and hurt their business.

### FIELD OF INVENTION

A hand held medical monitoring device that will allow you to check your blood levels high or low in an emergency.

### MYPERSONALLAB DESCRIPTION

MyPersonalLab will be a hand-held blood monitoring devise, small enough in size so that it can be taken with you, wherever you go. It will be able to monitor various blood electrolytes and other measures of blood components, the knowledge of which can help an individual manage a disease state, or help a doctor diagnose a medical problem. Fundamentally, it will function similar to a blood glucose monitor . . . the user will pierce their skin to obtain a small blood sample, apply it to a test strip, then insert the strip into the devise. The user will then wait a short while until the desired results are displayed. The devise will utilize a micro-process to evaluate the following electrolytes: Calcium, Magnesium, Potassium, Sodium, Chloride, & Phosphate, in addition to Blood pH and Blood Plasma Volume. The devise will have a feature that will allow you to download the data to a laptop or a PC, send it to a central website, or even e-mail results to your doctor. As you can see, this devise has great potential for improving the quality of life of its user.

### SUMMARY OF THE INVENTION

MyPersonalLab will be a hand-held blood monitoring devise, small enough in size so that it can be taken with you, wherever you go. MyPersonalLab to determine the quantitative value of various blood electrolytes to diagnose and treat a multitude of metabolic diseases much faster and therefore alleviating a patient's suffering that much faster.

### BREIF DESCRIPTION OF THE DRAWING

FIGS. 1-24

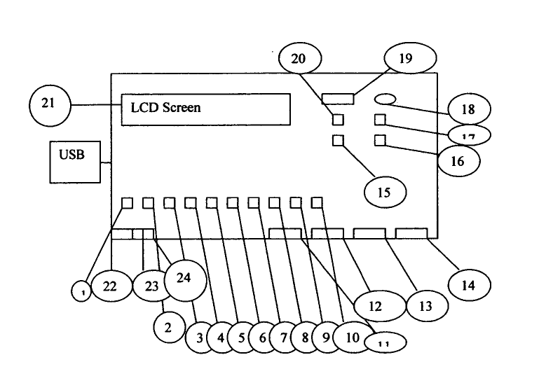

1) Calcium
2) Magnesium
3) Potassium
4) Sodium
5) Chloride
6) Phosphate Plasma
8) Blood PH
9) AST
10) ALT

```
Buttons on the right are:
      18 19
      On /Off
Internet Connection / Download / upload
      15 16 17
Four buttons on the bottom are:
Send to - Save As - Print - Copy To
11 12 13 14
Function on Left bottom is of stick insert for blood test
Buttons right next to stick insert is, Stick Insert GO/Results
LCDScreen 22 23/24
  21
```

### DETAILED DESCRIPTION OF THE EMBEDDED DIGITAL SYSTEM

A safe device that will allow the public to test there chemistry blood level from anywhere. This unit will have sensors to detect high or low levels of Calcium Blood PH, Potassium, Magnesium, AST, ALT, Sodium, Chloride, and Phosphate Plasma Included test results for the following: will utilize a micro-process to evaluate in whole blood, in mill equivalents, the following electrolytes are: Calcium, Magnesium, Potassium, Sodium, and Chloride & Phosphate. Also it will provide values of blood PH and Blood Plasma Volume, along with liver enzymes, with this device in hand you will be able to send your results to your doctor via email. The website will have a member login to where you can store all your data until you get home to print it out or save it to disk.

1. I (Melanie S Amaral) claim: All rights to MyPersonalLab Medical Monitoring Device. And including; all content within the device such as hardware, software, functions, features, and all website rights to MyPersonalLab.com.
